(19) United States
(12) Patent Application Publication
(10) Pub. No.: US 2026/0171232 A1
(43) Pub. Date: Jun. 18, 2026

(54) CONTEXT-AWARE INTERACTIVE EXPLORATION OF MAINTENANCE SERVICE REPORTS

(71) Applicant: KONINKLIJKE PHILIPS N.V., EINDHOVEN (NL)

(72) Inventors: Milosh STOLIKJ, EINDHOVEN (NL); Bart Albertus Gerardus VAN DER VELDEN, EINDHOVEN (NL); Qi GAO, EINDHOVEN (NL); Mauro BARBIERI, EINDHOVEN (NL); Johannes Henricus Maria KORST, EINDHOVEN (NL); Marc André PETERS, VELDHOVEN (NL)

(21) Appl. No.: 18/717,137

(22) PCT Filed: Dec. 7, 2022
(86) PCT No.: PCT/EP2022/084775

§ 371 Date: Jun. 6, 2024

Related U.S. Application Data
(60) Provisional application No. 63/288,700, filed on Dec. 13, 2021.

(51) Int. Cl. G16H 40/40

(52) U.S. Cl. CPC G16H 40/40

(57) ABSTRACT

A non-transitory computer readable medium (107, 127) stores a database of historical service reports (130) on previous service cases for medical devices (120); and instructions readable and executable by at least one electronic processor (101, 113) to receive a query providing details of a current service case for a medical device undergoing service; retrieve, from the database and based on the query, one or more historical service reports; analyze the retrieved historical service reports to identify information of the 2023/110577 retrieved historical service reports belonging to facets (132) of a set of facets; and display, on a display device (105), a user interface (UI) (138) configured to present a set of facet windows (136) corresponding to the set of facets with each facet window configured to present a summary of the information identified as belonging to the corresponding facet.

### FIELD

The following relates generally to the medical device maintenance arts, medical imaging device maintenance arts, medical device maintenance visualization arts, and related arts.

### BACKGROUND

A medical system (e.g., a medical imaging system) may occasionally malfunction (i.e., fail to work properly). One or multiple service engineers (SEs) then are assigned to diagnose and resolve the issue. The SEs can perform the diagnosis remotely and/or on site. During diagnosis, they can interact with several entities that can assist them during system troubleshooting, and for identifying the best course of action to resolve the issue. For example, they may decide to contact the customer to get a description of the problem, inspect the log files of the medical imaging system, to identify relevant diagnostic information such as error messages, search through historical service records to find similar service cases and identify how they were resolved, search through technical documentation for possible root causes and potential solutions, and so forth.

After the troubleshooting, the SE should be able to determine the root cause of the problem and identify the right service solution. The service solution can be executed remotely (e.g., a software update, remote calibration) or on-site when some parts need to be replaced.

The SEs are often under time pressure to perform the troubleshooting and decide on the most appropriate service action. Usually they need to collect and examine relevant information from various sources for that. The technical experience of SEs also differs. It can be beneficial to look at the previous successful cases (i.e., the problem was resolved by performing the right service action) from other engineers. These data are available in the form of historical service reports. However, there can be tens of thousands or more such service reports, and often due to the complexity of the problem and the medical system as well as the amount of information reported in these thousands or tens of thousands of service reports for historical service cases, it is challenging for the SEs to quickly identify the most relevant cases for the current problem, and to quickly review the most relevant information from the service reports for these cases.

The following discloses certain improvements to overcome these problems and others.

### SUMMARY

In one aspect, a non-transitory computer readable medium stores a database of historical service reports on previous service cases for medical devices; and instructions readable and executable by at least one electronic processor to receive a query providing details of a current service case for a medical device undergoing service; retrieve, from the database and based on the query, one or more historical service reports; analyze the retrieved historical service reports to identify information of the retrieved historical service reports belonging to facets of a set of facets; and display, on a display device, a user interface (UI) configured to present a set of facet windows corresponding to the set of facets with each facet window configured to present a summary of the information identified as belonging to the corresponding facet.

In another aspect, a non-transitory computer readable medium stores a database of historical service reports on previous service cases for medical devices; and instructions readable and executable by at least one electronic processor to retrieve, from the database and based on a received query providing details of a current service case for a medical device undergoing service, one or more historical service reports; analyze the retrieved historical service reports to identify information of the retrieved historical service reports belonging to facets of a set of facets; display, on a display device, a UI configured to present a set of facet windows corresponding to the set of facets with each facet window configured to present a summary of the information identified as belonging to the corresponding facet; receive, via at least one user input device, an input selecting an element of the information presented in a facet window; and adjust the facet window based on the selection.

In another aspect, a method of generating one or more facet windows for display during a servicing session of the medical device includes analyzing retrieved historical service reports to identify information of the retrieved historical service reports belonging to facets of a set of facets; displaying, on a display device, a UI configured to present a set of facet windows corresponding to the set of facets with each facet window configured to present a summary of the information identified as belonging to the corresponding facet; receiving, via at least one user input device, an input selecting an element of the information presented in a facet window ; and adjusting the facet window based on the selection.

One advantage resides in providing a tool for efficiently exploring a database of historical service cases during a servicing session of a medical device.

Another advantage resides in showing a visual summary of service cases to a SE in response to a search query.

Another advantage resides in showing visual representations of different stages of a workflow of a historical servicing session of service cases to a SE.

Another advantage resides in suggesting service actions for a SE to perform during a servicing session of a medical device.

A given embodiment may provide none, one, two, more, or all of the foregoing advantages, and/or may provide other advantages as will become apparent to one of ordinary skill in the art upon reading and understanding the present disclosure.

### BRIEF DESCRIPTION OF THE DRAWINGS

The disclosure may take form in various components and arrangements of components, and in various steps and arrangements of steps. The drawings are only for purposes of illustrating the preferred embodiments and are not to be construed as limiting the disclosure.

FIG. 1 diagrammatically illustrates an illustrative system for servicing medical device in accordance with the present disclosure.

FIG. 2 shows exemplary flow chart operations of the system of FIG. 1.

FIG. 3 shows an example user interface generated by of the system of FIG. 1.

### DETAILED DESCRIPTION

The following relates to an improved user interface and associated backend for searching historical service cases during a service call. In existing UI designs for reviewing historical service reports, the service engineer (SE) enters a search query and the historical service case reports matching that query are displayed in a summary list, which may include information such as match area, % match, or so forth. Such user interfaces provide the SE with little guidance to identify information relevant to the current case in the returned historical service reports.

In the disclosed improved UI, the returned historical cases are analyzed to identify elements of “facets”, i.e. categories, in the historical cases. Examples of such facets can include: “Case symptoms”, “Error codes”, “Service actions”, “Subsystems”, and “Replaced parts”, by way of non-limiting illustrative example. Elements of the historical cases belonging to the various facets are identified in various ways depending on the nature of the facet. For example, error codes are suitably identified using a database (DB) or list of predefined error codes. Symptoms and service actions are suitably identified on the basis of analysis by natural language processing (NLP) and/or keyword searching of respective freeform “Symptoms” and “Service Actions” text entry fields of the service report forms used to generate the service reports making up the historical case reports. Replaced parts may be identified using a predefined part numbers DB or by NLP of a freeform “Replaced Parts” text entry field.

With the elements of the various facets identified in the returned historical service reports, a facet window (or “widget”, or other display pane or the like) for each facet is constructed and displayed (though a given facet window might be hidden at any given time, for example the user may be able to selectively minimize or expand the facet windows). The detailed layout and information displayed for each facet window is tailored to the nature of the information collected for that facet. The information displayed may for some facets include statistical information assessed over all returned historical service cases. For example, an error codes window may list the error codes (or the most frequently occurring error codes) in the returned historical cases along with, for each listed error code, the number (or fraction) of the historical service cases that include that error code.

The SE can refine the search by selecting/deselecting specific elements displayed in the facet windows. This could include a “negative” selection of an element to exclude all historical service cases including that element; or a “positive” selection of an element to retain only those historical service cases that include that element. Each time the SE refines the search in such a way, the search results are updated and the analysis to determine the facet content is updated based on the updated service results. Where the update removes some historical service cases, the update can include recomputing the statistics for the reduced set of historical service cases. This provides a highly interactive way for the SE to explore the historical service cases and cull the original list (which may typically include hundreds or even thousands of returned historical cases) to a usable list of a few most relevant historical case reports. Furthermore, the identified elements for the various facets provides an intuitive summary of the content of those case reports.

In some embodiments, various elements of the facets may be weighted based on business or other priorities (e.g. service actions that do not involve parts replacement may be more heavily weighted to encourage consideration of solutions that avoid costly unnecessary part replacement), frequency of occurrence, or so forth.

In some embodiments, a co-replacement parts list (predefined or extracted from the search results or from a larger database of historical cases) is used to generate annotations indicating parts that are most frequently co-replaced with parts identified in the Parts facet.

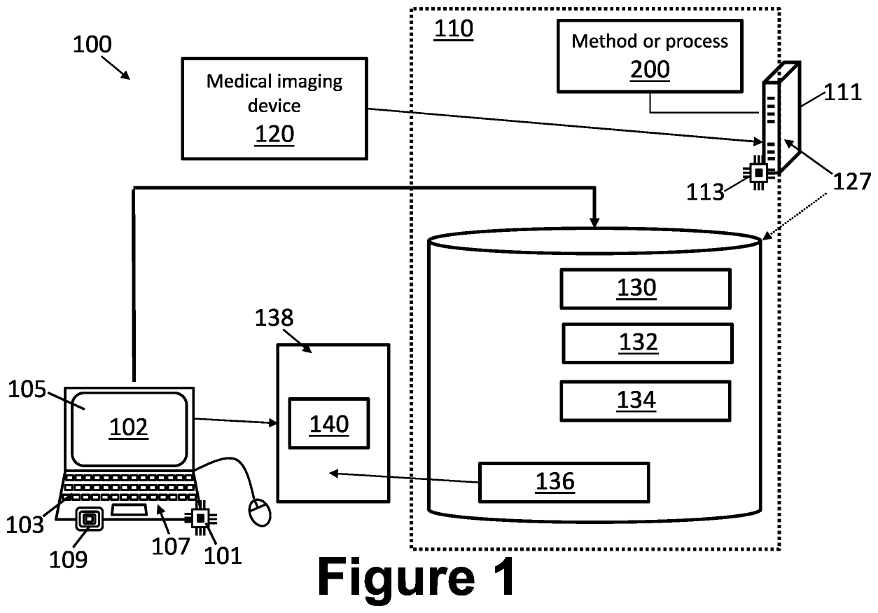

With reference to FIG. 1, an illustrative servicing support system 100 for supporting a service engineer in servicing an electronic device 120 (e.g., a medical imaging device-also referred to as a medical device, an imaging device, imaging scanner, and variants thereof) is diagrammatically shown. By way of some non-limiting illustrative examples, the medical imaging device under service may be a magnetic resonance imaging (MRI) scanner, a computed tomography (CT) scanner, a positron emission tomography (PET) scanner, a gamma camera for performing single photon emission computed tomography (SPECT), an interventional radiology (IR) device, or so forth. (More generally, the disclosed approach can be applied in conjunction with any type of computerized device for which historical service reports are routinely maintained in a searchable database, e.g., the approach could be applied to a commercial airliner, radiation therapy device, or so forth). As shown in FIG. 1, the servicing support system 100 includes, or is accessible by, a service device 102 that may for example be a workstation or electronic processing device used by a user (e.g., a service engineer (SE), such as a remote SE (RSE) or a field SE (FSE)). The service device 102 may for example be a portable device such as a notebook computer that is carried or accessed by an RSE. The service device 102 can be a desktop computer or a personal device, such as a mobile computer system such as a laptop or smart device. In other embodiments, the service device 102 may be an imaging system controller or computer integral with or operatively connected with the imaging device undergoing service (e.g., at a medical facility). As another example, the service device 102 may be a portable computer (e.g., notebook computer, tablet computer, or so forth) carried by a RSE performing diagnosis of a fault with the imaging device and ordering of parts. In another example, the service device 102 may be the controller computer of the imaging device under service, or a computer based at the hospital. In other embodiments, the service device may be a mobile device such as a cellular telephone (cellphone) or tablet computer.

The service device 102 includes a display device 105 via which alerts generated by predictive failure models are displayed, along with likely root cause and service action recommendation information as disclosed herein. The service device 102 also preferably allows the service engineer to interact with the servicing support system via at least one user input device 103 such a mouse, keyboard, or touchscreen. The service device further includes an electronic processer 101 and non-transitory storage medium 107 (internal components which are diagrammatically indicated in FIG. 1). The non-transitory storage medium 107 stores instructions which are readable and executable by the electronic processor 101 for interfacing with the servicing support system 100. The service device 102 also includes a communication interface 109 to communicate with a backend server or processing device 111, which typically implements the computational aspects of the servicing support system 100 (e.g., the server 111 has the processing power for implementing computationally complex aspects of the servicing support system 100). Such communication interfaces 109 include, for example, a wired and/or wireless Ethernet interface (e.g., in the case in which the service device 102 is a RSE workstation); or in the case in which the service device 102 is a portable FSE device the interface 109 may be a wireless Wi-Fi or 4G/5G interface or the like for connection to the Internet and/or an intranet. Some aspects of the servicing support system 100 may also be implemented by cloud processing or other remote processing (that is, the server computer 111 may be embodied as a cloud-based computing resource comprising a plurality of interconnected servers).

In illustrative FIG. 1, the servicing support system further includes a backend 110 (e.g., implemented and/or owned by the imaging device vendor or leased by the vendor from a cloud computing service provider; or in other embodiments the servicing support system 100 may be implemented and/or owned by a third party maintenance provider different from the imaging device vendor). The illustrative backend 110 optionally receives log data (e.g., a machine log automatically generated by the medical imaging device 120, a service log for the medical imaging device 120, and/or so forth) on a continuous or occasional basis (e.g., in some setups the imaging device 120 uploads machine log entries to the backend 110 on a daily basis). The backend processing for performing predictive fault modeling and (as disclosed herein) maintenance service analyses is performed on the backend server 111 equipped with an electronic processor 113 (diagrammatically indicated internal component). The server 111 is equipped with non-transitory storage medium 127 (internal components which are diagrammatically indicated in FIG. 1). While a single server computer is shown, it will be appreciated that the backend 110 may more generally be implemented on a single server computer, or a server cluster, or a cloud computing resource comprising ad hoc-interconnected server computers, or so forth. Furthermore, while FIG. 1 shows a single medical imaging device 120, more generally the database backend 110 will receive log data from many medical imaging devices (e.g., tens, hundreds, or more imaging devices) and performs the disclosed processing for a medical imaging device undergoing servicing using the log data generated by that device.

The non-transitory computer readable medium 127 stores historical service reports 130 on previous service cases for medical devices (including the medical device 120). For example, the service device 102 may have an application program loaded thereon that provides a service report entry user interface via which a SE enters a service report, that is, a report summarizing/describing a service case, and upon finalizing of the service report it is stored in the database of historical service reports 130. Over time, the database can accumulate hundreds, thousands, tens of thousands, or more historical service reports 130. In some embodiments, the service report entry user interface may provide various data entry fields, such as fields for entering information on the make, model, or other information about the medical device 120, a field for entering symptoms of the malfunction of the medical device 120, a field for entering service actions performed, a field for entering replaced part identifications, and/or so forth. The detailed format of the service report entry user interface can vary; for example, one embodiment may include separate fields for service actions and replaced parts, while another embodiment may provide a single field for entry of service actions with expectation that the SE will enter any replaced part identifications in conjunction with the associated part-replacement service action. Moreover, the various fields of the service report entry user interface can be of various formats, such as freeform text entry fields, drop-down lists, and/or so forth. It is also contemplated that when filing a finalized service case report, a validator or the like may be run on the content of the service report to detect obvious informalities (such as erroneous part numbers) and/or to suggest language conforming with a standard notation (for example, suggesting use of a standardized name for a particular service action). The validator can be run prior to storage of the finalized report in the database, so that the historical service reports 130 have improved accuracy and uniformity in terminology.

The non-transitory storage medium 127 also stores a set of facets 132 (i.e., “categories”) of the historical service reports 130. The facets 132 can include, for example, a “case symptoms” facet, an error codes facet, a service actions facet, a replaced parts facet, and so forth. Moreover, the non-transitory storage medium 127 also stores a co-replacement parts list 134 that lists parts of the medical device 120 that are most frequently co-replaced with other parts of the medical device 120. The set of facets 132 can, for example, comprise a predefined data structure for each facet defining the format of information corresponding to that facet. For example, if a part number always has a certain format, e.g. “AAANNNN” where “AAA” denotes three alphabetic letters and “NNNN” denotes four digits, then the format “AAANNNN” can be stored in the data structure for the replaced parts facet. Similarly, if information for a given facet is expected to be extracted from a specific field or fields of the service report, then the data structure for that facet can store an identification of that field or those fields. The data structures of the set of facets 132 can also store information about how the information corresponding to each facet is to be displayed. This enables the information associated with each facet to be displayed in a manner appropriate for that facet.

The non-transitory storage medium 127 also stores instructions executable by the electronic processor 113 of the backend server 111 to perform a method 200 of generating one or more facet windows 136 for display during a servicing session of the medical device 120 (e.g., a medical imaging device 120). It is noted that while FIG. 1 shows the set of facets 132 as distinct from the method 200, in some embodiments some or all of the information making up the set of facets 132 may be hard-coded in the instructions executable to perform the method 200. For example, each facet may be implemented as one or more executable subroutines, function calls, and/or other coding units implementing the extraction of information for that facet from the reports and/or the display of the facet window for that facet.

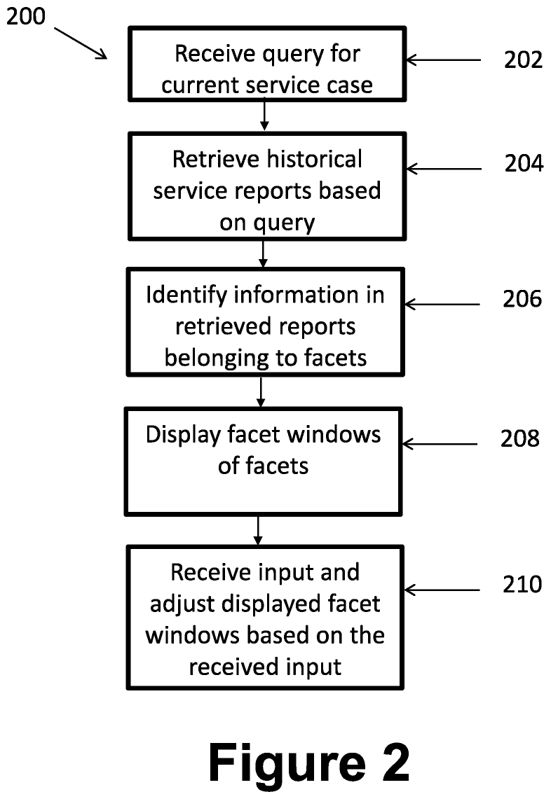

With continuing reference to FIG. 1 and further reference to FIG. 2, an illustrative embodiment of the method 200 executable by the electronic processor 113 of the backend server 111 is diagrammatically shown as a flowchart. In some examples, the method 200 may be performed at least in part by cloud processing.

At an operation 202, a query providing details of a current service case for the medical device 120 is provided by a SE via the service device 102, and the query is received at the backend server 111. At an operation 204, one or more historical service reports 130 are retrieved based on the query. (Typically, the operation 204 will retrieve dozens, hundreds, or even thousands or more historical service reports, though the number will depend on the content of the database and the nature/scope of the query). At an operation 206, the (typically large number of) retrieved historical service reports 130 are analyzed to identify information of the retrieved historical service reports 130 belonging to facets 132 of the set of facets 132.

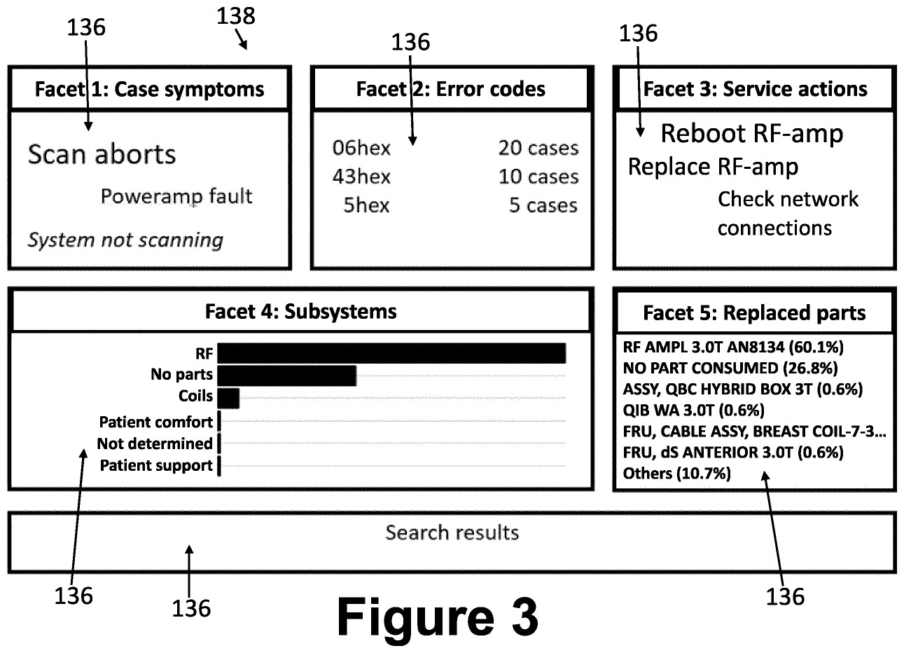

At an operation 208, a user interface (UI) 138 is displayed on the display device 105 of the service device 102. The UI 138 is configured to present a set of facet windows 136 corresponding to the set of facets 132 with each facet window 136 configured to present a summary of the information identified as belonging to the corresponding facet 134. As discussed, the formatting of each facet window 136 will, in general, be tailored to the information associated with that facet 132. Hence, various facet windows 136 may variously display lists of elements, word clouds (for example with words more frequently occurring in the reports shown with larger font size), bar graphs or other graphical representations of information, and/or so forth (see FIG. 3 for some examples). The UI 138 enables the SE to select the presented subset of the facet windows 136.

At an operation 210, an input selecting an element of the information presented in a facet window 136 is received at the backend server 111 and is provided by the SE via the user input device 103 of the service device 102. The facet window 136 can then be adjusted based on the selection of the element. In one example, the adjusting operation 210 includes displaying additional details related to the selected element of the facet window 136. In another example, the adjusting operation 210 includes culling the retrieved historical service reports 130 by removing all service reports 130 containing the selected element of the facet window 136. The analysis operation 206 is then repeated on the culled retrieved historical service reports 130, for example recalculating any statistics over the reports for the culled set of reports, and the displayed facet windows 136 are updated based on the repeated analysis operation 206.

In some embodiments, one or more elements of the facets 134 are weighted with one or more predetermined weights to decide elements and method to display in the facet window 136. In other embodiments, the weights can be adjusted by the user, to decide elements and method of display in the facet window 136. In other embodiments, annotations 140 are generated using the co-replacement parts list 134. The annotations 140 are indicative of one or more parts of the medical device 120 that are most frequently co-replaced with parts listed in a parts facet window 136.

In one particular example, the set of facets 132 includes a case symptoms facet and a service actions facet. The information belonging to the case symptoms facet 132 include case symptoms contained in the retrieved historical service reports 130 and a quantification of occurrences of each case symptom in the retrieved historical service reports 130. The information belonging to the service actions facet 132 comprise service actions contained in the retrieved historical service reports 130 and a quantification of occurrences of each service action in the retrieved historical service reports 130. The facet window 136 corresponding to the case symptoms facet 132 presents a tag cloud of the symptoms with an emphasis of each presented symptom indicating the quantification of occurrences of the symptom in the retrieved historical service cases 130. The facet window 136 corresponding to the service action facet 132 presents a tag cloud of the service actions with an emphasis of each presented service action indicating the quantification of occurrences of the service action in the retrieved historical service cases 130.

In another particular example, the set of facets 132 includes an error codes facet 132 and a replaced parts facet 132. The information belonging to the error codes facet 132 comprise error codes contained in the retrieved historical service reports 130 and a quantification of occurrences of each error code in the retrieved historical service reports 130. The information belonging to the replaced parts facet 132 comprise replaced parts contained in the retrieved historical service reports 130 and a quantification of occurrences of each replaced part in the retrieved historical service reports 130. The facet window 136 corresponding to the error codes facet 132 presents a ranked list of at least a portion of the error codes ranked by the corresponding quantification of occurrences of the error code in the retrieved historical service reports 130. The facet window 136 corresponding to the replaced parts facet 132 presents a ranked list of at least a portion of the replaced parts ranked by the corresponding quantification of occurrences of the replaced part in the retrieved historical service reports 130.

In some embodiments, the information displayed in the facet windows 136 (e.g., case symptoms, error codes, service actions, replaced parts, and so forth) can be used to determine a root cause, or a potential solution, to the case symptom(s) causing the issue with the medical device 120. To do so, the electronic processor 113 of the backend server 111 can use an artificial intelligence (AI) algorithm to analyze the information displayed in the facet windows 136 to determine the root cause (or potential solution) of the issue with the medical device 120. Such AI algorithms can be suitably trained on labeled historical training data which may also have been generated using the content of the facet windows for earlier (i.e. historical) cases and labeled with root causes previously determined by service engineers.

FIG. 3 shows an example of the UI 138. The summarized information is presented in multiple facet windows 136, with each facet window 136 containing information related to a specific semantic category. As used herein, “semantic category” refers to distinct information types that may be present in a case: symptoms, error messages, subsystems, part descriptions, part type numbers, service actions etc. The UI 138 enables the SE to search and refine the search results without the need for textual input. In the illustrative example of FIG. 3, the UI 138 includes five facet windows 136: Facet 1 presenting case symptoms information extracted from the historical service cases; Facet 2 presenting error codes extracted from the historical service cases; Facet 3 presenting service actions information extracted from the historical service cases; Facet 4 presenting subsystems referred to in the historical service cases; and Facet 5 presenting replaced parts information extracted from the historical service cases. These are merely nonlimiting illustrative examples. Moreover, it is noted that in some embodiments the user can minimize a facet window or (in some embodiments) close a facet window entirely—hence in such embodiments all five illustrative facet windows may not be presented on the display at any given time.

### EXAMPLES

The following describes some nonlimiting illustrative example embodiments of the system 100 and the method 200 in more detail. Let c denote a service case, C denote a set of service cases, q denote a search query, comprising terms and logical operators, and C(q) denote the search results from query q, where C(q)⊆C. Let a denote a textual entry by a SE, or a text description of a case activity, A denote a set of textual descriptions, and A(c) denote a set of textual descriptions that comprise a case, with A(c)⊆A. Let the semantic class of an activity be denoted by s(a). Let t denote a term (e.g. word or sequence of words), T denote a set of terms, and T(a) denote the set of terms in an activity, with T(a)⊆T.

The SE engages the interactive UI 138 by either manually entering a search query q, or by selecting a textual field from the existing populated textual fields in the service case that the SE is working on. The interactive UI 138 then performs the search query, and, along with showing (a subset of) the search results C(q), it shows one or more facets 132. Each facet 132 corresponds to one semantic class of activities s. The set of relevant activities from all search results is given by RA={a|c∈C(q), a ∈A(c), s(a)=s}. Then, the facet 132 shows a subset of the terms found in those activities: TF⊂{t|t∈T(a), a∈RA}. The subset can be created by e.g., using Term Frequency-Inverse Document Frequency to rank all terms in T(a), and selecting the top-K scored terms as the subset TF. K is an integer value, typically set in relation to the type and the size of the facet. The terms can be displayed using different text formatting such as font size, font colour, bold, underline etc.

The SE may interact with any facet window 136. Hovering over any term shows the number of service cases in C(q) that contain that term. Each facet window 136 allows addition or removal of terms from the search query q to refine the search results. For example, addition of term t in the query q would result in query q′=[q, AND(t)]. Similarly, removal of term t in the query q would result in query q′=[q, NOT(t)]. In one example, this interaction can be supported by providing, on the UI 138, distinct icons for selection and deletion, for example, a check mark (✓) and an X mark (X) respectively. These icons are hidden by default and made visible when the pointer hovers over a term or in case of a touch screen, when the SE taps on a term. Based on whether the SE clicks the selection or the deletion icons, the term is highlighted, for example with an emphasis or a strikeout respectively, to give visual feedback to the user. In another example, this interaction can be supported by providing, on the UI 138, columns, lists or other such relevant areas into which the SE can drag a term or group of terms from the facet window 136 and drop it into these areas. These areas could be assigned with different purposes, for example, as terms to be removed, terms that are similar in meaning, terms that have similar user-defined attributes, etc.

After the SE chooses the set of terms using the techniques stated above and clicks a button to refine the search, the UI 138 performs another query and retrieves the refined results. As a result of such an action, a new set of cases C'(q) is created, and all facets are re-computed on the new set. If the system's hardware is fast enough, this querying can also be performed instantaneously, i.e., as, and when the SE is selecting or removing terms from the UI 138, and the results can be displayed immediately.

This interaction process continues until the SE has found what they are looking for. The output data in the results may or may not be coupled with other types of visualizations such as charts and graphs or just a list of search results presented as hyperlinks to documents alongside the facets. In such cases, these associated visualizations change synchronously or asynchronously with the facet windows 136.

The UI 138 can also provide configuration options to enable or disable spelling correction, synonym selection and other such universal operations. Additionally, if synonym selection is enabled, the UI 138 can provide an option to include or exclude some or all the synonyms for the next search iteration. In this way, the SE can select the synonyms that are relevant and exclude the ones that are not relevant for subsequent search queries.

In some embodiments, each term t is associated with one or more weights. One example of a term weight could be the score returned by the search engine that determines the similarity of the service case to the query entered by the SE. Typical examples of such scores are TF-IDF score, Okapi BM25 etc. Another example is the service cases priority, ranging from critical to scheduled maintenance. The user can change the weight of terms in the facet windows 136 by interacting with the facet windows 136. The term weight may impact whether the term is displayed in the facet or not, or on the manner how the terms is visualized.

In some embodiments, a semantic class may represent symptoms exhibited by the medical device 120. These symptoms are typically captured in natural language, as either the description of the behaviour of the medical device 120 given by the customer, or by an observation done by the SE in the field. To extract terms from this type of input, the SE would typically use a technique such as term frequency-inverse document frequency, with a variable number of n-grams.

In some embodiments, a semantic class may represent error codes found in the data, or displayed in the UI 138. The set of error codes is pre-defined, and can be retrieved from an external system such as a database. Each error code is treated as a single term.

In some embodiments, a semantic class may represent the subsystem of the medical device 120. This subsystem may be determined by a machine learning algorithm.

In some embodiments, a semantic class may represent the service actions that may be taken by the SE.

In some embodiments, a semantic class may represent the replaced parts in historical cases. The replaced parts can be shown by their part type number, or by the description, written in natural language. The entire description would represent a single term.

In some embodiments, when looking at the details of the case (e.g., similar historical service reports 130), or during life inspection of the medical device 120, the SE could observe a part that may malfunction. The SE can be presented a view of parts that are frequently replaced during the same call, together with the suspected part using the co-replacement parts list 134. This may hint the SE to inspect one or more of such parts as well. It can also help the SE in having a closer look at some of these parts and find out which part is malfunctioning, which can help in determining other parts that should be replaced, also it can help in avoiding unnecessary replacements by comparing the functioning of a small set of relevant parts.

A non-transitory storage medium includes any medium for storing or transmitting information in a form readable by a machine (e.g., a computer). For instance, a machine-readable medium includes read only memory (“ROM”), solid state drive (SSD), flash memory, or other electronic storage medium; a hard disk drive, RAID array, or other magnetic disk storage media; an optical disk or other optical storage media; or so forth.

The methods illustrated throughout the specification, may be implemented as instructions stored on a non-transitory storage medium and read and executed by a computer or other electronic processor.

The disclosure has been described with reference to the preferred embodiments. Modifications and alterations may occur to others upon reading and understanding the preceding detailed description. It is intended that the exemplary embodiment be construed as including all such modifications and alterations insofar as they come within the scope of the appended claims or the equivalents thereof.

1. A non-transitory computer readable medium storing:
a database of historical service reports on previous service cases for medical devices; and
instructions readable and executable by at least one electronic processor to:
receive a query providing details of a current service case for a medical device undergoing service;
retrieve, from the database and based on the query, one or more historical service reports;
analyze the retrieved historical service reports to identify information of the retrieved historical service reports belonging to facets of a set of facets; and
display, on a display device, a user interface (UI) configured to present a set of facet windows corresponding to the set of facets with each facet window configured to present a summary of the information identified as belonging to the corresponding facet.

2. The non-transitory computer readable medium of claim 1, wherein the facets include one or more of case symptoms, error codes, service actions, and replaced parts.

3. The non-transitory computer readable medium of claim 2, wherein the facets comprise one or more case symptoms, and one or more of error codes, service actions, and replaced parts; and the instructions further include instructions to:
analyze the one or more case symptoms and the one or more of error codes, service actions, and replaced parts identified as belonging to a corresponding facet to determine a root cause or potential solution to the one or more case symptoms.

4. The non-transitory computer readable medium of claim 1, wherein the UI enables a user to select the presented subset of the facet windows.

5. The non-transitory computer readable medium of claim 1, wherein the instructions further include:
receiving, via at least one user input device an input selecting an element of the information presented in a facet window; and
adjusting the facet window based on the selection.

6. The non-transitory computer readable medium of claim 5, wherein the adjusting includes:
displaying additional details related to the selected element of the facet window.

7. The non-transitory computer readable medium of claim 5, wherein the adjusting includes:
culling the retrieved historical service reports by removing all service reports containing the selected element of the facet window;
repeating the analysis on the culled retrieved historical service reports; and
updating the displayed facet windows based on the repeated analysis.

8. The non-transitory computer readable medium of claim 1, wherein the instructions further include:
weighting one or more elements of the facets with one or more weights to decide elements to display in the facet window.

9. The non-transitory computer readable medium of claim 1, wherein the instructions further include:
generate, using a co-replacement parts list annotations indicative of part of the medical device that are most frequently co-replaced with parts listed in a parts facet window.

10. The non-transitory computer readable medium of claim 1, wherein:
the set of facets includes a case symptoms facet and a service actions facet,
the information belonging to the case symptoms facet comprise case symptoms contained in the retrieved historical service reports and a quantification of occurrences of each case symptom in the retrieved historical service reports,
the information belonging to the service actions facet comprise service actions contained in the retrieved historical service reports and a quantification of occurrences of each service action in the retrieved historical service reports,
the facet window corresponding to the case symptoms facet presents a tag cloud of the symptoms with an emphasis of each presented symptom indicating the quantification of occurrences of the symptom in the retrieved historical service cases; and
the facet window corresponding to the service action facet presents a tag cloud of the service actions with an emphasis of each presented service action indicating the quantification of occurrences of the service action in the retrieved historical service cases.

11. The non-transitory computer readable medium of claim 1, wherein:
the set of facets includes an error codes facet and a replaced parts facet,
the information belonging to the error codes facet comprise error codes contained in the retrieved historical service reports and a quantification of occurrences of each error code in the retrieved historical service reports,
the information belonging to the replaced parts facet comprise replaced parts contained in the retrieved historical service reports and a quantification of occurrences of each replaced part in the retrieved historical service reports,
the facet window corresponding to the error codes facet presents a ranked list of at least a portion of the error codes ranked by the corresponding quantification of occurrences of the error code in the retrieved historical service reports; and
the facet window corresponding to the replaced parts facet presents a ranked list of at least a portion of the replaced parts ranked by the corresponding quantification of occurrences of the replaced part in the retrieved historical service reports.

12. A non-transitory computer readable medium storing:
a database of historical service reports on previous service cases for medical devices; and
instructions readable and executable by at least one electronic processor to:
retrieve, from the database and based on a received query providing details of a current service case for a medical device undergoing service, one or more historical service reports;
analyze the retrieved historical service reports to identify information of the retrieved historical service reports belonging to facets of a set of facets;
display, on a display device a user interface (UI) configured to present a set of facet windows corresponding to the set of facets with each facet window configured to present a summary of the information identified as belonging to the corresponding facet;
receive, via at least one user input device an input selecting an element of the information presented in a facet window and
adjust the facet window based on the selection.

13. The non-transitory computer readable medium of claim 12, wherein the UI enables a user to select the presented subset of the facet windows.

14. The non-transitory computer readable medium of claim 13, wherein the adjusting includes:
displaying additional details related to the selected element of the facet window.

15. The non-transitory computer readable medium of claim 13, wherein the adjusting includes:
culling the retrieved historical service reports by removing all service reports containing the selected element of the facet window;
repeating the analysis on the culled retrieved historical service reports; and
updating the displayed facet windows based on the repeated analysis.

16. The non-transitory computer readable medium of claim 12, wherein the instructions further include:
weighting one or more elements of the facets with one or more predetermined weights to decide elements to display in the facet window.

17. The non-transitory computer readable medium of claim 12, wherein the instructions further include:
generate, using a co-replacement parts list annotations indicative of part of the medical device that are most frequently co-replaced with parts listed in a parts facet window.

18. The non-transitory computer readable medium of claim 12, wherein the facets include one or more of case symptoms, error codes, service actions, and replaced parts.

19. The non-transitory computer readable medium of claim 12, wherein the medical device comprises a medical imaging device.

20. A method of generating one or more facet windows for display during a servicing session of the medical device the method comprising:
analyzing retrieved historical service reports to identify information of the retrieved historical service reports belonging to facets of a set of facets;
displaying, on a display device a user interface (UI) configured to present a set of facet windows corresponding to the set of facets with each facet window configured to present a summary of the information identified as belonging to the corresponding facet;
receiving, via at least one user input device an input selecting an element of the information presented in a facet window; and
adjusting the facet window based on the selection.
